(19) United States
(12) Patent Application Publication
(10) Pub. No.: US 2006/0136009 A1
(43) Pub. Date: Jun. 22, 2006

(54) Dental/medical anxiety/phobia remediation protocol

(76) Inventors: Edward R. Staffel, San Antonio, TX (US); G. Blake Holloway, Kerrville, TX (US)

Correspondence Address: Charles W. Hanor, P.C., PO Box 91319, San Antonio, TX, 78209, US

(21) Appl. No.: 11/021,405

(22) Filed: Dec. 22, 2004

(51) Int. Cl. A61N 1/34

(52) U.S. Cl. 607/46

(57) ABSTRACT

A protocol or procedure is provided for lowering sympathetic nervous system arousal in a person in order to prepare that person for a medical or dental procedure. First, a therapeutic dosage of one or more neurotransmitter supplements, such as a gamma aminobutyric acid formulation, a tryptophan-derived neurotransmitter, and dehydroepiandrosterone are sublingually administered to the patient. After a period of time for the formula dosage to take effect, gelled electrodes are placed adjacent or below the mastoid. The gelled electrodes are connected to a cranial electrotherapy stimulation device that administers a sub-sensation level current to the patient. Also, a noise dampening headset is placed on the patient and a neuroacoustic entrainment recording or program is played. Then, the medical or dental procedure is performed.

### FIELD OF THE INVENTION

This invention relates to medical and dental care, and more particularly to an anxiety reduction protocol designed to lower sympathetic nervous system arousal in a person.

### BACKGROUND

Individuals who have unwarranted and inappropriate fears and anxiety about medical and dental treatments often refuse needed medical and dental care. Even when such individuals agree to undergo a medical or dental procedure, their fears and anxiety can make the experience unnecessarily unpleasant. There is therefore a need for a method of providing individuals relief and remediation from anxiety and phobia in preparation for a medical or dental procedure.

### SUMMARY

An object of the present invention is to lower sympathetic nervous system arousal in a person (i.e., relax the person) in order to prepare that person for a medical or dental procedure. In furtherance of that object, a method of preparing a patient for a medical or dental procedure is provided that comprises the steps of: seating the person in a comfortable reclining chair; sublingually administering a therapeutic dosage of a neurotransmitter supplement to the patient; administering cranial electrotherapy stimulation to the patient; and administering a neuroacoustic entrainment program to the patient to promote lower-frequency brainwave patterns. The neuroacoustic entrainment program preferably comprises either a recording embedded with a monaural beat or a stereo recording comprising signals of two different frequencies presented separately and simultaneously to each ear. This induces the brain to perceive a phantom frequency (i.e., a binaural beat) equal to the difference between the two frequencies presented to the ears. Finally, the medical practitioner performs the medical or dental procedure on the patient. The method may further involve administering additional dosages of neurotransmitter supplements to the patient during the dental procedure.

Preferably, the person is first seated, then administered a therapeutic dosage of a neurotransmitter supplement, and thereafter concomitantly administered cranial electrotherapy stimulation and a neuroacoustic entrainment program. Different embodiments of the method comprise administering one or more of the following: a gamma aminobutyric acid formulation, a tryptophan-derived neurotransmitter, and dehydroepiandrosterone. The step of administering cranial electrotherapy stimulation preferably comprises delivering current at a sub-sensation intensity level transcranially via gelled electrodes placed adjacent or below the mastoid. The neuroacoustic entrainment program is preferably administered to the patient through a noise dampening headset, and the program preferably comprises multiple layered binaural signals that promote multiple lower-frequency brainwave patterns, wherein the binaural signals are blended with instrumental music or sounds of nature.

The combined regimen of treatments targets the neurobiology of stress and arousal, enhancing the ability of the central nervous system to restore homeostasis between the sympathetic and parasympathetic nervous system, which in turn reduces anxiety and phobia in the medical or dental setting.

The method is preferably performed in a doctor office, hospital or outpatient setting. But the method may also be performed in an ambulatory vehicle or home. These and other suitable applications, modifications, and enhancements of the invention will be readily apparent to those skilled in the art from the following detailed description taken in conjunction with the annexed sheets of drawings, which illustrate the invention.

### BRIEF DESCRIPTION OF THE DRAWINGS

FIG. 1 is a flow chart illustrating one embodiment of a method of lowering sympathetic nervous system arousal in a person in order to prepare that person for a medical or dental procedure.

FIG. 2 is a diagram illustrating a person reclining in a chair receiving concomitant administration of cranial electro-stimulation, neuroacoustic entrainment, and a neurotransmitter supplement.

### DETAILED DESCRIPTION

Although the following specific details describe aspects of various embodiments of the invention, persons reasonably skilled in the art will recognize that various changes may be made in the details of the invention without departing from its spirit and scope as defined in the appended claims. Therefore, it should be understood that, unless otherwise specified, this invention is not to be limited to the specific details shown and described herein.

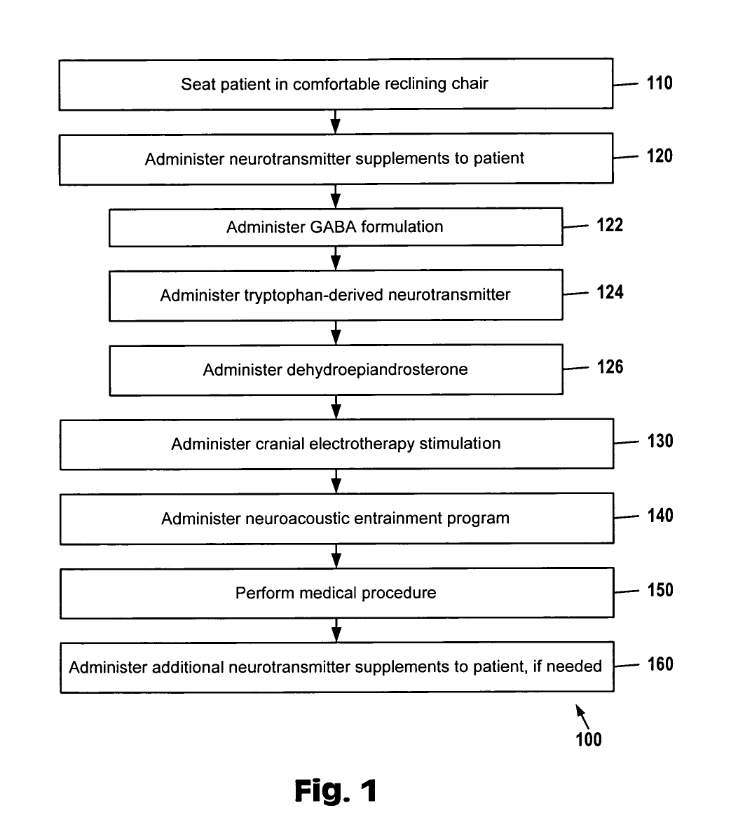

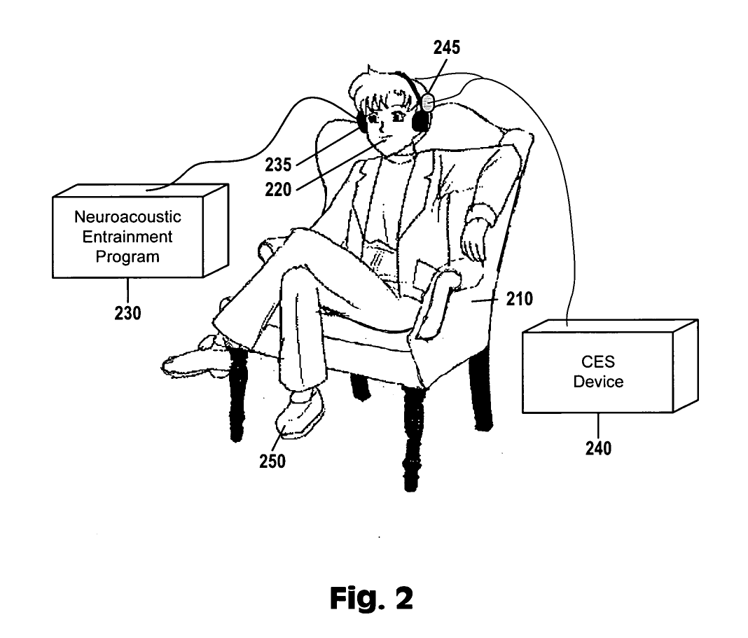

FIG. 1 is a flow chart illustrating one embodiment of a method 100 for lowering sympathetic nervous system arousal in a person in order to prepare that person for a medical or dental procedure. FIG. 2 illustrates a patient 250 being prepared by that method for the medical or dental procedure. The patient 250 should arrive at the clinic approximately thirty to forty minutes prior to the planned dental or medical procedure. In step 110, the patient 250 is placed in a comfortable reclining chair 210. In step 120, a therapeutic dosage of one or more neurotransmitter supplements 220, such as a gamma aminobutyric acid formulation 122, a tryptophan-derived neurotransmitter 124, and dehydroepiandrosterone 126 are sublingually administered to the patient 250. In step 130, after a period of time for the formula dosage to take effect, gelled electrodes 245 are placed adjacent or below the mastoid. The gelled electrodes 245 are connected to a cranial electrotherapy stimulation (CES) device 240 that administers a sub-sensation level current to the patient 250. An optional timer (not shown) causes the CES device 240 to administer the current to the patient 250 on a continuous or intermittent basis. In step 140, a noise dampening headset 235 is placed on the patient 250 and a neuroacoustic entrainment recording or program 230 is played. In step 150, the medical or dental procedure is performed.

In one embodiment, the cranial electrotherapy stimulation, neuroacoustic entrainment program, and neurotransmitter supplementation regimens are administered only before, but not during, the dental or medical procedure. In another embodiment, one or more of the cranial electrotherapy stimulation, neuroacoustic entrainment program, and neurotransmitter supplementation regimens continue to be applied during the dental or medical procedure, particularly if the procedure is lengthy.

Applicants have discovered that the administration of neurotransmitter supplements, cranial electrotherapy stimulation, and neuroacoustic entrainment have complementary effects on the stimulation of neurotransmitters (i.e., chemical substances that transmit nerve impulses across a synapse) associated with relaxation and a sense of well-being. The following paragraphs describe the meaning of, additional details of, various purposes of, and benefits resulting from, administering these complementary regimens to patients about to undergo a medical or dental procedure.

The Neurobiology of Stress and Arousal

Several systems of the human body participate in responses to stress, including the sensory thalamus, the sensory cortex, the hippocampus, the amygdala, the hypothalamic-pituitary-adrenal axis (HPA axis), and the sympathetic nervous system. In response to sensory stimulus that could indicate a danger, the sensory thalamus communicates with the amygdala through two pathways. The thalamus communicates directly and immediately through a sub-cortical pathway to the amygdala, without any intervening cognition. The thalamus also communicates indirectly and more slowly with the amygdala through the cortex and hippocampus. The cortex, which is involved with cognition, and the hippocampus, which stores conscious memories and provides contextual information, tells the amygdala whether a perceived threat is real.

The amygdala stores implicit memories such as conditioned responses to aversive stimuli and emotional memories associated with fear. It comprises several physically close but functionally distinct nuclei. The basolateral complex of the amygdala processes inputs from the sensory system and perceives and evaluates the significance of a threat posed by that sensory system input. Its main output is the central nucleus of the amygdala, which is involved in emotional arousal. The central nucleus, in turn, sends fear-signaling impulses to the hypothalamus.

In response to fear-signaling impulses, the hypothalamus releases a stress hormone called corticotrophin-releasing factor (CRF), which in turn stimulates the pituitary gland to release the stress hormone adrenocorticotropic hormone (ACTH), which in turn stimulates the adrenal cortex to release coricosteroids into the blood stream. Corticosteroids, such as cortisol, are important to developing the body's fight or flight response to danger.

In response to fear-signaling impulses, the hypothalamus also activates the sympathetic nervous system (SNS). High SNS arousal is responsible for the uncomfortable symptoms of anxiety. The SNS prepares the body for immediate and vigorous defensive action by tightening muscles, constricting blood vessels, increasing the heart rate, metabolism, and blood pressure and sugar levels, dilating the eye's pupils and the lungs' trachea and bronchi, shunting blood to the skeletal muscles, liver, brain, and heart, stimulating the adrenal glands, and stimulating the liver to convert glycogen to glucose. The parasympathetic nervous system (PNS), by contrast, slows the heartbeat, constricts the bronchi, and generally restores the body to a normal state.

Both the SNS and PNS operate through neurotransmitters that communicate along the neural pathways of the SNS and PNS. The SNS and PNS each comprise (1) preganglionic neurons that connect the central nervous system (CNS) to ganglions of the body; and (2) postganglionic neurons that run from the ganglions to the effector organ.

Preganglionic sympathetic neurons release the excitatory neurotransmitter acetylchlorine, and postganglionic sympathetic neurons release noradrenaline (also called norepinephrine). Because each preganglionic sympathetic neuron usually synapses with many postganglionic neurons, and because some of the neurons release noradrenaline and adrenaline (also called epinephrine) directly into the blood, activation of the SNS generally affects several body functions simultaneously.

As noted above, the neural systems of the body that participate in the physiology of stress and anxiety use neurotransmitters to communicate. Two of the most prominent anxiety neurotransmitters are gamma aminobutyric acid (GABA) and serotonin. GABA is the primary inhibitory neurotransmitter of the central nervous system. GABA receptors are found on 25-40% of the synapses of the brain. When GABA binds to a GABA receptor, it opens a chlorine channel allowing negatively charged chlorine ions into the interior of the nerve cell. This, in turn, polarizes the neuron which inhibits further presynaptic release of neurotransmitters. By inhibiting neural firing, GABA suppresses anxiety-related messages from reaching the cortex.

When subjected to prolonged stress or anxiety, the brain depletes its available store of GABA and other inhibitory neurotransmitters. This can culminate in a full-blown anxiety or panic attack, accompanied by excessive sweating, trembling, muscle tension, weakness, disorientation, breathing difficulty, fear, and other symptoms.

Serotonin (also known as 5-hydroxytryptophan) is a neurotransmitter produced by neurons located in the locus coeruleus and raphe nuclei of the brain. The locus coeruleus and raphe nuclei innervate the thalamus, cerebral cortex, and hippocampus. Low levels of serotonin are associated with depression, anxiety, sleeplessness, impulsive behavior, aggression, and violent activity.

Neurotransmitter Supplementation

The use of a therapeutic dosage of neurotransmitter supplement in preferred embodiments of the present invention has an anxiolytic effect on the brain by relieving anxiety and reducing tension on the patient.

In one embodiment, the patient is administered a therapeutic dosage of an amino acid formula comprising: 1-6 mg of magnesium (in the form of magnesium taurinate), 50-200 mg of GABA, 10-80 mg of glycine, 10-40 mg of N-Acetyl-L-Tyrosine, and 5-20 mg of taurine (also in the form of magnesium taurinate). This formula contains three of the main inhibitory neurotransmitters plus N-Acetyl L-Tyrosine, which is a precursor of norepinipherine, another neurotransmitter. Together, the formula has an inhibitory effect on the neurons of the brain, and therefore reduces sympathetic nervous system arousal. The formula is preferably administered in the form of one or more sublingual tablets or sublingual liptropic sprays. By administering the formula sublingually, the formula is absorbed directly into the bloodstream through the blood vessels under the tongue and in the cheeks, allowing quick entry of the formula into the system.

In a second embodiment, the patient is administered a neurotransmitter supplementation formula comprising a combination of vitamin B6, vitamin C, bioflavonoids, and tryptophan or L-5-hydroxytryptophan (L-5-HTP). A therapeutic dosage of one such combination comprises 25-400 mg of L-Theanine, 10-150 mg of L 5 Hydroxy-Tryptophan, 20-100 mg of Pyridoxal-5′-phosphate (i.e., vitamin B6), and 100-800 mg of ascorbic acid (i.e., vitamin C). The patient's own response to the regimen may dictate the actual dosage rate. Like the amino acid formula described above, the serotonin-boosting formula is preferably administered sublingually, through one or more tablets or lipotropic sprays. Alternatively, it is administered in the form of a enterically-coated capsule meant to be swallowed. The enteric coating enables the capsules to bypass enzymes in the stomach that would convert the L-5-HTP to serotonin prematurely, before reaching the central nervous system.

Tryptophan-derived neurotransmitters serve as the precursor for the synthesis of serotonin (i.e., 5-hydroxytryptamine or 5-HT) and melatonin (N-acetyl-5-methoxytryptamine). Vitamin B6 is the co-factor for enzymes that convert L-tryptophan to serotonin, and Vitamin C catalyzes the hydroxylation of tryptophan to serotonin.

In a third embodiment, the patient is administered dehydroepiandrosterone (DHEA). DHEA, a hormone naturally secreted by the adrenal gland, is a signaler that reduces the level of the stress hormone cortisol in the body. This addition to the formulary provides some systemic relief for individuals under chronic stress and/or anxiety.

Preferred embodiments administer a combination of some or all of the foregoing supplements.

Neuroacoustic entrainment

The incorporation of neuroacoustic entrainment into preferred embodiments of the present invention is intended to enhance certain brainwave frequencies that boost the brain's own production and transmission of neurotransmitters.

The electrochemical activity of the brain produces measurable electromagnetic wave forms. Electroencephalographs (EEGs) of the brain can quantify this activity in terms of amplitude and frequency. Research has associated different brainwave frequencies with different mental states. At frequencies of between 13 and 40 Hz (i.e., Beta brainwaves), the mind is active, alert and able to focus on details. Beta brainwave states are associated with conversation and competitive physical activities. Between 8 and 13 Hz (i.e., Alpha brainwave states), the mind is more relaxed and reflective. Alpha brainwave states are associated with creativity, contemplation, and visualization. Theta brainwave states, which operate in the range of 4 to 8 Hz, are associated with dreaming, intense creativity, visualization ability, meditation, and out-of-body experiences. The brain cells reset their sodium and potassium ratios in the Theta state, which helps explain why sleep is important to healthy mental function. Delta brainwave states, which operate in the range of 0.5 to 4 Hz, are associated with unconsciousness, very deep and dreamless sleep, and long term memories. Some very experienced and disciplined meditative individuals are able to train their minds to operate in the Delta state while conscious.

A normal healthy human brain cycles through each of these brainwave states throughout the day and night. And because not all parts of the brain are equally active at all times, the brain typically operates in several brainwave states at the same time. A person's state of mind or level of consciousness is typically associated with the dominant brainwave state.

It is also believed that some brainwave frequencies promote the production and transmission of certain neurotransmitters more than other brainwave frequencies. One study associated a 10 Hz brainwave pattern with enhanced production and turnover of serotonin. For this reason, it is believed that by entraining the brain to a given frequency, the production of some neurotransmitters can be enhanced.

There are various methods of entraining brainwave states, including disciplined meditation, chanting, and hypnosis. Most methods entrain the brain to operate in a given brainwave state by subjecting it to a repeated stimulus, such as pulses of sound, volume modulation, monaural beats, binaural beats, or flashes of light. A constant, repeated 10 Hz stimulus applied to the brain can stimulate a 10 Hz brainwave state. This phenomenon is called the “frequency following response.”

In the preferred embodiments of the present invention, a noise dampening headset is placed over the patient's ears and connected to a neuroacoustic entrainment recording. In one embodiment, the recording preferably includes a sound or melody that is volume modulated at frequencies designed to train the brain to enter an alpha or theta brainwave state.

In another embodiment, the recording is designed to make the brain perceive one or more binaural beats. Binaural beats occur when signals of two different frequencies are presented separately, one to each ear. Each ear is hardwired to an olivary nucleus (a sound processing center of the brain) in the corresponding hemisphere of the brain. The brain, in trying to reconcile the different noises it hears from each ear, perceives a “binaural beat.” If a frequency of 100 Hz is presented to one ear, and a frequency of 105 Hz is presented to the other, the brain will perceive a binaural beat of 5 Hz, and may ultimately be entrained to resonate at the binaural beat frequency.

In preferred embodiments of the invention, the neuroacoustic entrainment program uses a recording that introduces multiple binaural frequencies, preferably in the form of harmonically layered and patterned binaural frequencies. In this manner, multiple frequency following responses are triggered in the brain at the same time. Furthermore, the binaural frequencies are preferably blended together with instrumental music, soothing sounds of rain, or nature sounds.

Cranial Electrotherapy Stimulation

The incorporation of cranial electrotherapy stimulation (CES) into preferred embodiments of the present invention is intended to stimulate a balance in the nervous system's release and transmission of excitatory and inhibitory classes of neurotransmitters. CES, also known as microcurrent electrical stimulation or transcranial microcurrent stimulation, is the application of low-level, pulsed electrical currents to the head. The current is preferably applied to the patient via gel electrodes on or below the mastoid, or alternatively at the central prefrontal area of the forehead and Inion at the back of the head.

The current is preferably provided in the form of sinusoidal, rectangular, or modified rectangular wave pulses. The frequency is set between 0.1 and 1,000 Hz, optionally superimposed on a carrier wave of up to 150 kHz. The waveform is preferably biphasic and bipolar and has a 20-50% duty cycle. The intensity of the current is set at a stable level below the sensation threshold (e.g., between 0.01 mA and 7 mA, more preferably about 0.1 mA). The CES device 240 should be able to adjust the voltage for varying levels of resistance in order to provide a stable level of current. CES is preferably applied for at least 30 minutes, and as long as 6 hours. The frequency pattern and current level may be adjusted from time to time to prevent physiological accommodation.

CES is thought to stimulate the vagus nerve, and thereby promote PNS dominance. CES also focuses current on the hypothalamic region of the brain, where it influences both the pre-synaptic release and post-synaptic reception of neurotransmitters. The current increases serotonin and endorphin levels in the brain and decreases the level of the stress hormone cortisol in the brain. It also promotes alpha brainwave states. It is believed to operate by stimulating the neurons of the brain to accelerate their manufacture and reception of both inhibitory and excitatory neurotransmitters at the same rate, so that they mutually inhibit each other's further production, restoring the neurotransmitters to pre-stress homeostasis. For example, CES applied to an anxious patient will slow down the patient's norepinephrine neurons and speed up the patient's endorphin neurons, bringing them into homeostatic balance. For this reason, CES is believed to have a more dramatic effect on anxious patients, who are out of balance, than relaxed patients who are already in a homeostatic balance.

Synergistic Benefits

The concomitant use of neuroacoustic entrainment, neurotransmitter supplementation, and electro-stimulation has many synergistic benefits. CES and neuroacoustic entrainment both promote homeostasis between the sympathetic and parasympathetic nervous systems, thereby lowering sympathetic system arousal. The use of CES together with neuroacoustic entrainment promotes relaxing alpha brainwave states more than might be achieved with neuoacoustic entrainment alone. The use of neurotransmitter supplements helps to prevent pre-synapse vesicle depletion that might otherwise occur with CES if sufficient precursor amino acids are not available. Furthermore, all three treatments increase the release inhibitory neurotransmitters. The restoration of neurochemical homeostais remediates the excitatory neurological events that form the basis of severe anxiety.

A conformational single blind study was conducted to gather data to support the experience of anxious dental patients who have used the studied protocol to help them undergo procedures that, in the past, caused them severe anxiety requiring sedation. A small sample of subjects with evoked anxiety were exposed to Cranial Electrotherapy Stimulation, Neuroacoustic Software and administered Amino Acid Supplements composed of GABA, 5HTP, Theanine with cofactors in the full protocol. This study subjected the subjects on a second day to all of the above components except the Non-Neuroacoustic Software (soothing music) was substituted for the Neuroacoustic Software, this was called the Sham treatment.

The research study was divided into two phases. Phase I included measurements of GSR and SEMG. Data collected from subjects in phase II included GSR readings and EEG taken at “cz”. All data were recorded using Thought Technologies BioGraph ProComp: Version 2.0. Channels Alpha, Theta, Beta and GSR were selected for analysis. The Anxiety Survey was a modified anxiety checklist, given post-evoked anxiety and then again post-treatment on both treatment days.

When comparing data subjects exposed to the full treatment showed, on average, a 30 point decrease in anxiety levels and a 4.47 microvolt decrease in the GSR recordings than sham treatment produced. The EEG data from the Neuroacoustic Software treatment as compared to the sham treatment showed an average decrease in the Beta brainwave power of −0.30 indicating a decrease in vigilance and an increase in relaxation. In addition, the Theta and Alpha brainwaves showed an increase in power, Theta +0.7 and Alpha +1.8. Both of these increases indicate the subject became more relaxed and restful. This is in comparison to the sham treatment that showed an increase in Beta (move vigilance), a −0.05 microvolt decrease in Theta (less restful) and a 1.34 microvolt difference in Alpha (less relaxed). As Theta and Alpha power increased subjects became more relaxed and therefore more likely to be less agitated during a medical and or dental procedure. The SEMG data was unstable and not included in the analysis.

Along with the brainwave and GSR data it is essential to look at the subjects decrease in perceived anxiety as indicated by a reduction in their anxiety score and their reflections after each days study. Subjects reflected that with both protocols they felt more relaxed but on the day they received the full protocol their mind was able to let go of their thoughts and they become much more at ease and at peace than on the day they received the sham Neuroacoustic Software. They reported that they felt better able to manage their stress and anxiety causing situations after the full treatment than with the partial treatment. Neuroacoustic Software when used with CES and Supplements reduces anxiety and increases relaxation better than when CES and Supplements are used alone.

Conclusion

From the above it can be appreciated that the treatment modalities of the present invention have therapeutic applications to individuals who react with acute anxiety to dental and other medical procedures. Other benefits and applications of the present invention will be apparent to persons of ordinary skill in the art.

As used in the claims below, the term “neurotransmitter supplement” refers not only to supplements of natural neurotransmitters, but also to chemical supplements used by the body to synthesize a neurotransmitter, inhibit the reuptake of a neurotransmitter, or have an effect similar to that of a natural neurotransmitter.

Although the foregoing specific details describe various embodiments of the invention, persons reasonably skilled in the art will recognize that various changes may be made in the details of the apparatus of this invention without departing from the spirit and scope of the invention as defined in the appended claims. Therefore, it should be understood that, unless otherwise specified, this invention is not to be limited to the specific details shown and described herein.

1. A method of relaxing a patient before and during a dental procedure comprising the steps of:
administering a therapeutic dosage of a neurotransmitter supplement to the patient;
administering cranial electrotherapy stimulation to the patient;
administering a neuroacoustic entrainment program to the patient to promote lower-frequency brainwave patterns, the neuroacoustic entrainment program comprising signals of two different frequencies presented separately and simultaneously to each ear, the program inducing the brain to perceive a phantom frequency equal to the difference between the two frequencies presented to the ears; and
performing the dental procedure on the patient.

2. The method of claim 1, wherein the step of administering a therapeutic dosage of a neurotransmitter supplement precedes the administration of cranial electrotherapy stimulation and a neuroacoustic entrainment program.

3. The method of claim 2, wherein the administration of cranial electrotherapy stimulation and a neuroacoustic entrainment program are done concomitantly.

4. The method of claim 1, further comprising the step of seating the person in a piece of furniture that supports a person in a reclining position.

5. The method of claim 4, wherein the person is first seated, then administered a therapeutic dosage of a neurotransmitter supplement, and thereafter administered cranial electrotherapy stimulation and a neuroacoustic entrainment program.

6. The method of claim 1, wherein the step of administering a therapeutic dosage of a neurotransmitter supplement precedes the steps of administering cranial electrotherapy stimulation and administering a neuroacoustic entrainment program.

7. The method of claim 1, wherein the neurotransmitter supplement is administered sublingually.

8. The method of claim 1, wherein the neurotransmitter supplement comprises a gamma aminobutyric acid formulation.

9. The method of claim 1, wherein the neurotransmitter supplement comprises tryptophan or a tryptophan derivative.

10. The method of claim 1, further comprising administering dehydroepiandrosterone to the patient.

11. The method of claim 1, wherein the step of administering cranial electrotherapy stimulation comprises delivering current transcranially via gelled electrodes placed adjacent or below the mastoid.

12. The method of claim 1, wherein the step of administering cranial electrotherapy stimulation comprises delivering current at a sub-sensation intensity level.

13. The method of claim 1, wherein the neuroacoustic entrainment program is administered to the patient through a noise dampening headset.

14. The method of claim 1, wherein the neuroacoustic entrainment program comprises multiple layered binaural signals that promote multiple lower-frequency brainwave patterns.

15. The method of claim 1, wherein the neuroacoustic entrainment program blends instrumental music or sounds of nature with the binaural signals.

16. The method of claim 1, further comprising administering additional dosages of neurotransmitter supplements to the patient during the dental procedure.

17. A method of lowering sympathetic nervous system arousal in a person in order to prepare that person for a medical or dental procedure, the method comprising the steps of:
administering a therapeutic dosage of a neurotransmitter supplement to the person;
administering cranial electrotherapy stimulation to the person; and
administering a neuroacoustic entrainment program to the person to promote lower-frequency brainwave patterns, the neuroacoustic entrainment program comprising binaural signals of two different frequencies presented separately and simultaneously to each ear, the program inducing the brain to perceive a phantom frequency equal to the difference between the two frequencies presented to the ears.
